(19) United States
(12) Patent Application Publication
(10) Pub. No.: US 2006/0084061 A1
(43) Pub. Date: Apr. 20, 2006

(54) Methods for blocking adipocyte differentiation and triglyceride accumulation with interleukin 12 p35 inhibitors

(76) Inventors: Eric G. Marcusson, San Diego, CA (US); Nicholas M. Dean, Olivenheim, CA (US)

Correspondence Address: ISIS PHARMACEUTICALS, INC.., 1896 RUTHERFORD RD., CARLSBAD, CA, 92008, US

(21) Appl. No.: 10/512,739

(22) PCT Filed: Jun. 10, 2003
(86) PCT No.: PCT/US03/18320

§ 371 Date: Oct. 27, 2004

(30) Foreign Application Priority Data
Jun. 11, 2002 (US) 60388074

(51) Int. Cl. C12Q 1/68

(52) U.S. Cl. 435/6

(57) ABSTRACT

Methods for blocking adipocyte differentiation and triglyceride accumulation with inhibitors of Interleukin 12 p35 and provided. Interleukin 12 p35 inhibitors of the present invention include small molecules, antibodies, peptides (including dominant negative peptides) and antisense compounds, including ribozymes, inhibitory RNA molecules including siRNA molecules and antisense oligonucleotides.

This application claims the benefit of U.S. Provisional Application No. 60/388,074, filed Jun. 11, 2002.

### BACKGROUND OF THE INVENTION

Obesity is known to be a major health risk throughout Europe and the United States leading to a number of potentially life threatening diseases. Obesity is usually defined as being about 20% above the mean adiposity. Lifelong obesity is associated with an excess number of adipocytes, presumably a genetically determined phenomenon. On the other hand, the obesity that begins in adult life develops against a background of larger—that is, hypertrophied—adipocytes, the number of which remains the same. An excessive recruitment and differentiation of preadipocytes into mature adipocytes is a characteristic of human obesity, which is a strong risk factor for Type 2 diabetes, certain cancers, and cardiovascular disease, including hypertension, atherosclerosis, and coronary artery disease. Obesity and insulin resistance share a complex relationship that gives rise to a range of metabolic disorders, including Type 2 diabetes. Obesity can itself engender insulin resistance. Reaven, G. M., Physiol. Rev., 1995, 75, 473-486. The most important consequence of obesity is type II (maturity-onset) diabetes, which is associated with normal or high level of circulating insulin and peripheral resistance to insulin's action. Most human obesity is associated with insulin resistance and leptin resistance. In fact obesity may have an even greater impact on insulin action than does diabetes itself. Sindelka et al., Physiol Res., 2002, 51, 85-91. Weight reduction usually ameliorates the glucose intolerance of type II diabetes, presumably owing to a decrease in the stimulus for insulin secretion by the pancreatic beta cells. Furthermore, it is believed that as the fat cells (adipocytes) accumulate triglycerides, they release free fatty acids. A flux of these fatty acids to the liver may be important in the cause of diabetes.

In addition to diet control, several methods of chemically treating obesity with pharmacologically active substances have been identified. However, these methods may cause other health problems. For example, caffeine- and amphetamine-based diet aids may be addictive and adversely affect other areas of health. The combination of fenfluramine and phentermine has been proven to cause heart valve disease.

Hyperlipidemia is an abnormally high concentration of lipids in the blood serum. The composition of the lipid pool in the circulation consists mostly of triglyceride (fatty acid esters of glycerol), cholesterol, and fatty acid esters of cholesterol. It is believed that as the fat cells (adipocytes) accumulate triglycerides, they release free fatty acids. Fatty acids are precursors to cholesterols. As such, a reduction of triglyceride synthesis effectively reduces cholesterol. Lipid molecules are generally bound to and are carried by specific proteins, known as apoproteins. Various combinations of different and specific lipids and apoproteins form lipoproteins. Lipoproteins can transport lipids and perform specific biological functions.

The form of hyperlipidemia characterized by excessively high triglyceride levels in plasma is called hypertriglyceridemia. Elevated triglycerides may be a consequence of other disease, such as untreated diabetes mellitus. Like cholesterol, high in triglyceride levels are detected by plasma measurements. These measurements should be made after an overnight food and alcohol fast. The National Cholesterol Education Program guidelines for triglycerides are (based on fasting triglyceride levels): Normal: Less than 150 mg/dL; Borderline-high: 150-199 mg/dL; High: 200-499 mg/dL; Very High: 500 mg/dL or higher.

Common pathological sequelae of hyperlipidemia include cardiovascular diseases or conditions including coronary artery disease, atherosclerosis, hypertension, thrombosis, and ischemic events (for example, myocardial infarction, cerebral stroke, and organ insufficiency). Insulin resistance is also associated with hypertriglyceremia. Sindelka et al., Physiol Res., 2002, 51, 85-91.

Various drugs are available which can lower serum lipid levels in human patients. For example, Lopid™ (available from Parke-Davis), and Tricor™ (available from Abbott), are effective in treating Type IV and V hyperlipidemias, with triglyceride levels being abnormally high. However, these drugs may cause many side effects, some of which are quite severe.

Syndrome X or Metabolic syndrome is a new term for a cluster of conditions, that, when occurring together, may indicate a predisposition to diabetes and cardiovascular disease. These symptoms, including high blood pressure, high triglycerides, decreased HDL and obesity, tend to appear together in some individuals.

Needed, therefore, are improved methods for blocking adipocyte differentiation and/or triglyceride accumulation.

It is now, surprisingly, discovered that an inhibitor of Interleukin 12 p35 is effective to block adipocyte differentiation and/or triglyceride accumulation. It is believed that these inhibitors will be useful in the prevention and treatment of diseases or conditions associated with high levels of triglycerides and with excess (i.e., higher than average) or unwanted numbers of adipocytes. These conditions include hypertriglyceridemia, hyperlipidemia, obesity, and sequelae of one or more of these conditions, including metabolic syndrome, diabetes, insulin resistance, and cardiovascular diseases and conditions including coronary artery disease, atherosclerosis, hypertension, thrombosis and ischemic events (for example, myocardial infarction, cerebral stroke, and organ insufficiency).

Cytokines are soluble factors produced by lymphocytes that regulate the survival, proliferation, differentiation, and homeostasis of cells involved in mediating the immune response. These factors do not only activate other lymphocytes, they also relay signals to non-lymphoid cells including macrophages, epithelial and stromal cells, creating a broad spectrum of cytokine activity that is critical to the maintenance of health. Consequently, much effort has been devoted to the study of these proteins.

Interleukin 12 (IL-12; formerly NKSF, for natural killer cell stimulatory factor, or CLMF, for cytotoxic lymphocyte maturation factor) is a cytokine produced by monocytes, macrophages, neutrophils, dendritic cells and antibody-producing B cells (Hall, Science, 1995, 268, 1432-1434) as well as keratinocytes and epidermoid carcinoma cell lines (Aragane et al., J. Immunol., 1994, 153, 5366-5372).

Interleukin 12 is responsible for activation of natural killer (NK) cells, T cells and induction of increased levels of interferon-gamma, a cytokine that helps to shape the immune response (Hall, Science, 1995, 268, 1432-1434). The combination of interferon-gamma and interleukin 12 sends a powerful signal to native precursor cells of the T helper lineage, shifting the immune system to a TH1-type immune response (Hall, Science, 1995, 268, 1432-1434). Generally, resistance to pathogens increases when interleukin 12 is present to drive a TH1 response (Hall, Science, 1995, 268, 1432-1434).

Interleukin 12 is unique among the cytokines because it is a disulfide-linked heterodimer composed of unrelated 40-kD (p40) and 35-kD (p35) subunits that are encoded by genes on separate chromosomes. The p35 and p40 subunits are localized to chromosomes 3 p12-3q13.2 and 5q31-q33 respectively (Sieburth et al., Genomics, 1992, 14, 59-62). The p35 subunit shares structural similarities with the cytokines interleukin 6 and granulocyte colony-stimulating factor (Merberg et al., Immunol. Today, 1992, 13, 77-78). Alternatively, the p40 subunit is structurally related to the interleukin 6 receptor (Gearing and Cosman, Cell, 1991, 66, 9-10).

Disclosed and claimed in U.S. Pat. No. 5,457,038 and PCT publication WO 92/05206 is a DNA sequence coding for interleukin 12 or a subunit thereof (Trinchieri et al., 1995; Trinchieri et al., 1992).

cDNAs for both subunits of interleukin 12 were cloned in 1991 from a lymphoblastoid B-cell line (Gubler et al., Proc. Natl. Acad. Sci. USA, 1991, 88, 4143-4147). Both subunits are required to obtain the biologically active heterodimer (Gubler et al., Proc. Natl. Acad. Sci. USA, 1991, 88, 4143-4147) and p35 is only secreted as part of the heterodimer (D'Andrea et al., J. Exp. Med., 1992, 176, 1387-1398) whereas p40 can be induced and secreted independently (Snijders et al., J. Immunol., 1996, 156, 1207-1212) and has no biological activity (Ling et al., J. Immunol., 1995, 154, 116-127). Bioactive murine and human single chain interleukin 12 fusion proteins expressed from retroviral constructs have been demonstrated to retain antitumor activity in vivo (Lieschke et al., Nat. Biotechnol., 1997, 15, 35-40).

Interleukin 12 has been found to be upregulated in vivo during murine lipopolysaccharide-induced endotoxemia and to stimulate the synthesis of interferon-gamma (Heinzel et al., Infect. Immun., 1994, 62, 4244-4249). However, pretreatment of the mice with anti-interleukin 12 antibodies caused a reduction in interferon-gamma levels after lipopolysaccharide injection (Heinzel et al., Infect. Immun., 1994, 62, 4244-4249).

Astrocyte-targeted expression of both interleukin 12 p35 and p40 genes in mice promoted the spontaneous development of a severe neuroimmunological disorder with many features resembling those of experimental allergic encephalomyelitis (EAE) (Pagenstecher et al., J. Immunol., 2000, 164, 4481-4492).

Analysis of the cytokine pattern expressed in situ in inhalant allergen patch test reactions of atopic dermatitis patients indicate increased expression of interleukin 12 p35 subunit. This upregulation of the p35 subunit may contribute to the observed switch of the cytokine secretion pattern (Grewe et al., J. Invest. Dermatol., 1995, 105, 407-410).

Structure-function analysis of the mouse interleukin 12 p35 subunit has indicated that p35 participates in both receptor binding and signaling (Zou et al., J. Biol. Chem., 1995, 270, 5864-5871).

Tone et al. have determined that the murine interleukin 12 p35 subunit has multiple transcription start sites and can initiate from either of two 5′ exons, resulting in mRNA isoforms with different 5′ untranslated regions (Tone et al., Eur. J. Immunol., 1996, 26, 1222-1227). In a separate study, the same research group found that p35 subunit mRNA was up-regulated by lipopolysaccharide stimulation of murine B cell lymphoma line A20 and in bone marrow-derived dendritic cells (Babik et al., J. Immunol., 1999, 162, 4069-4078). Four p35 subunit mRNA isoforms were found in both cell types and transcription was found to initiate from alternate positions after lipopolysaccharide stimulation. Further regulation of p35 subunit was observed at the translational level in addition to the transcriptional level (Babik et al., J. Immunol., 1999, 162, 4069-4078).

Interleukin 12 p35 and p40 antisense probes were used in in situ hybridization studies that identified enhanced interleukin 12 transcription in the gastric mucosa of pediatric patients with Crohn's disease (Berrebi et al., Am. J. Pathol., 1998, 152, 667-672).

The homodimer of the interleukin 12 p40 subunit (also known as (p40)₂) has been found to be a very potent inhibitor of interleukin 12 activity (Gillessen et al., Eur. J. Immunol., 1995, 25, 200-206; Ling et al., J. Immunol., 1995, 154, 116-127) and functions by binding to the interleukin 12 receptor (Ling et al., J. Immunol., 1995, 154, 116-127). The p40 homodimer was used as an antagonist of interleukin 12 in investigative treatments of mice with cyclophosphamide-induced diabetes and found to dampen islet formation (Rothe et al., Diabetologia, 1997, 40, 641-646). The p40 homodimer was found to inhibit the antitumor activity of the interleukin 12 heterodimer and the induction of interferon-gamma in murine bladder carcinoma in vivo. (Chen et al., J. Immunol., 1997, 159, 351-359).

Carter et al. have produced antibodies to recombinant human interleukin 12 which have been demonstrated to neutralize the biological activity of interleukin 12. However, essentially all antibodies were generated to the p40 subunit, possibly due to conformational limitations of the intact interleukin 12 heterodimer (Carter et al., Hybridoma, 1997, 16, 363-369). Larsson et al. have reported an immunoassay that only recognized the bioactive human interleukin 12 heterodimer and not the individual p35 and p40 subunits (Larsson and Linden, Cytokine, 1998, 10, 786-789). Disclosed and claimed in PCT publication WO 99/37682 are anti-human interleukin 12 antibodies that are characterized by specificity to the interleukin 12 heterodimer but do not bind to the interleukin 12 p40 subunit (Gately and Presky, 1999).

In investigations of the mechanisms of anti-inflammatory effects of corticosteroids budesonide was found to inhibit production of bioactive interleukin 12 in human monocytes (Larsson and Linden, Cytokine, 1998, 10, 786-789).

Pentoxifylline, a non-specific phosphodiesterase inhibitor exhibited complex effects on the expression of interleukin 12. The production of interleukin 12 p35 subunit was inhibited but, the production of the p40 subunit was enhanced (Marcinkiewicz et al., Immunopharmacology, 2000, 49, 335-343).

The involvement of interleukin 12 p35 subunit in immune system regulation and as well as viral and bacterial infections make it a potentially useful therapeutic target for intervention in autoimmune diseases. Currently, inhibitors of the interleukin 12 p35 subunit and/or the interleukin 12 heterodimer include the p40 homodimer, antibodies and small molecules such as corticosteroids and the phosphodiesterase inhibitor pentoxyfylline.

It is now surprisingly discovered that inhibitors of Interleukin 12 p35 can be used to block differentiation of preadipocytes to adipocytes and to block triglyceride accumulation in adipocytes.

### SUMMARY OF THE INVENTION

It is now surprisingly discovered that inhibitors of Interleukin 12 p35 can be used to block differentiation of preadipocytes to adipocytes and to block triglyceride accumulation in adipocytes. Methods for inhibiting the differentiation of an adipocyte cell or for inhibiting lipid accumulation, particularly triglyceride accumulation, in a cell by contacting the cell with an inhibitor of Interleukin 12 p35 activity or expression are provided. Methods for treating, preventing or delaying the onset of diseases or conditions associated with adipocyte differentiation, excess adipocytes or lipid accumulation, particularly triglyceride accumulation or high triglyceride levels, are also provided. The inhibitor of Interleukin 12 p35 may be a small molecule, antibody, peptide and/or antisense compound.

Additional advantages and aspects of the present invention are apparent in the following detailed description and claims.

### DETAILED DESCRIPTION OF THE INVENTION

It is now surprisingly discovered that the inhibition of Interleukin 12 p35 can reduce or prevent adipocyte differentiation and triglyceride accumulation.

An adipocyte cell is a connective tissue cell specialized for the synthesis and storage of fat. During differentiation from pre-adipocytes to adipocytes, numerous changes occur, including accumulation of triglycerides as lipid droplets, secretion of several hormones and autocrine factors (e.g., leptin and TNF-a), and changes in gene expression. Mature adipocyte cells are swollen with globules of triglycerides; increased triglyceride content is a well established marker of adipocyte differentiation in culture. Mature adipocytes are also characterized by a number of molecular markers that are not present in pre-adipocytes. During adipocyte differentiation, the gene expression patterns in adipocytes change considerably. “Hallmark” or marker genes for adipocyte differentiation include adipocyte lipid binding protein 2 (aP2), glucose transporter 4 (GLUT4) and hormone sensitive lipase (HSL). The products of these genes play important roles in the uptake of glucose and the metabolism and utilization of fats. The presence of one, or preferably more than one, more preferably all of these gene products is indicative of mature adipocytes, i.e., of differentiation of adipocytes from preadipocyte cells.

In one embodiment, inhibitors of Interleukin 12 p35 may be administered to reduce or prevent adipocyte differentiation and/or triglyceride accumulation. Furthermore, conditions associated with adipocyte differentiation, triglyceride accumulation and excess adiposity may also be treated by the administration of a Interleukin 12 p35 inhibitor. These conditions include, for example, obesity, hyperlipidemia, and associated conditions and/or sequelae such as cardiovascular disease, metabolic syndrome, diabetes and/or insulin resistance. As used herein, “treatment” includes prophylactic as well as therapeutic use, i.e., treatment of a disease or condition includes prevention as well as delay of onset of the disease or condition.

In a broad embodiment, the Interleukin 12 p35 protein of a mammal may be inhibited by the administering to the mammal a therapeutically effective amount of an inhibitor of Interleukin 12 p35. As used herein, a Interleukin 12 p35 inhibitor is a compound that inhibits Interleukin 12 p35 expression, levels, or activity. As used herein, “inhibit” may be partial or complete reduction in the amount or activity of Interleukin 12 p35 to a level at or below that found under normal physiological conditions if used prophylactically, or below the existing (pre-treatment) levels if used in treatment of an active or acute condition. In one embodiment, the activity or amount of Interleukin 12 p35 is inhibited by about 10%. Preferably, the activity or amount of Interleukin 12 p35 is inhibited by about 30%. More preferably, the activity or amount of Interleukin 12 p35 is inhibited by 50% or more. In one embodiment, the reduction of the expression of targets may be measured in adipose, liver, blood or other tissue of the mammal. Preferably, the cells being inhibited contain therein a nucleic acid molecule encoding for a Interleukin 12 p35 protein and/or the Interleukin 12 p35 protein itself. As used herein, a mammal is a warm-blooded vertebrate animal, which includes a human.

Any inhibitor of Interleukin 12 p35 may be employed in accordance with the present invention. Compounds useful as Interleukin 12 p35 inhibitors include compound that act on the Interleukin 12 p35 protein to directly inhibit Interleukin 12 p35 function or activity, as well as compounds which indirectly inhibit Interleukin 12 p35 by reducing amounts of Interleukin 12 p35, e.g., by reducing expression of the gene encoding Interleukin 12 p35 via interference with transcription, translation or processing of the mRNA encoding Interleukin 12 p35. Inhibitors of Interleukin 12 p35 also include compounds that bind to Interleukin 12 p35 and inhibit its function, including ability to bind substrate or receptor molecules and/or any enzymatic or other activity that Interleukin 12 p35 may have. Thus inhibitors of Interleukin 12 p35 include small molecules, preferably organic small molecule compounds; antibodies; peptides and peptide fragments, particularly Interleukin 12 p35 dominant negative peptides and fragments, and the like. Inhibitors of Interleukin 12 p35 also include compounds which inhibit the expression or reduce the levels of Interleukin 12 p35, including small molecules, antibodies, peptides and peptide fragments, nucleic acids and the like which are designed to bind to a particular target nucleic acid and thereby inhibiting its expression. In one embodiment, Interleukin 12 p35 inhibitors used in accordance with the present invention are antisense compounds. Non-limiting examples of antisense compounds in accordance with the present invention include ribozymes; short inhibitory RNAs (siRNAs); long double-stranded RNAs, antisense oligonucleotides; antisense oligonucleotide mimetics such as peptide nucleic acid (PNA), morpholino compounds and locked nucleic acids (LNA); external guide sequence (EGS); oligonucleotides (oligozymes) and other short catalytic RNAs or catalytic oligonucleotides which hybridize to the target nucleic acid and modulate its expression, and mixtures thereof. Antisense inhibitors of Interleukin 12 p35 are disclosed in U.S. Pat. No. 6,399,379, which is incorporated herein in its entirety.

In one embodiment, small molecules are administered as Interleukin 12 p35 inhibitors in accordance with the present invention. Pentoxifylline, a non-specific phosphodiesterase inhibitor, inhibits production of interleukin 12 p35 subunit. Marcinkiewicz et al., Immunopharmacology, 2000, 49, 335-343. Libraries of small organic molecules may be obtained commercially, for example from ChemBridge Corp. in San Diego, Calif. or LION Bioscience, Inc. (formerly Trega Biosciences) in San Diego, Calif. Libraries of small molecules may also be prepared according to standard methods that are well known in the art. An appropriate screening or assaying for inhibitors of the desired molecule is essential to finding inhibitors with the desired selectivity and specificity, and such screening and assaying may be readily practiced by one of ordinary skill in the art.

In another embodiment, Interleukin 12 p35 inhibitors are antibodies or fragments thereof. These antibodies or fragments thereof may selectively bind to Interleukin 12 p35 and in so doing, selectively inhibit or interfere with the Interleukin 12 p35 polypeptide, preferably with the activity thereof. Standard methods for preparation of monoclonal and polyclonal antibodies and active fragments thereof are well known in the art. Antibody fragments, particularly Fab fragments and other fragments which retain epitope-binding capacity and specificity are also well known, as are chimeric antibodies, such as “humanized” antibodies, in which structural (not determining specificity for antigen) regions of the antibody are replaced with analogous or similar regions from another species. Thus antibodies generated in mice can be “humanized” to reduce negative effects which may occur upon administration to human mammals. Chimeric antibodies are now accepted therapeutic modalities with several now on the market. The present invention therefore includes use of antibody inhibitors of Interleukin 12 p35 which include F(ab′)2, Fab, Fv and Fd antibody fragments, chimeric antibodies in which one or more regions have been replaced by homologous human or non-human portions, and single chain antibodies. U.S. Pat. No. 6,150,401 discloses techniques for antibodies specific for a protein, for example Interleukin 12 p35. These techniques may be employed to produce inhibiting antibodies specific for Interleukin 12 p35. The disclosure of U.S. Pat. No. 6,150,401 is incorporated in its entirety herein by reference. Antibodies to Interleukin 12 p35 are well known and are commercially available, for example from Santa Cruz Biotechnology (Santa Cruz Calif.) Catalog #SC-1280).

In other embodiments, the present invention provides use of Interleukin 12 p35 inhibitors which are peptides, for example dominant negative Interleukin 12 p35 polypeptides. A dominant negative polypeptide is an inactive variant or fragment of a protein which competes with or otherwise interferes with the active protein, reducing the function or effect of the normal active protein. If the target protein is an enzyme, dominant negatives may include polypeptides which have an inactive or absent catalytic domain, so that the polypeptide binds to the substrate but does not phosphorylate it, or polypeptides which have a catalytic domain with reduced enzymatic activity or reduced affinity for the substrate. One of ordinary skill in the art can use standard and accepted mutagenesis techniques to generate dominant negative polypeptides. For example, one of ordinary skill in the art can use the nucleotide sequence of Interleukin 12 p35 along with standard techniques for site-directed mutagenesis, scanning mutagenesis, partial deletions, truncations, and other such methods known in the art. For examples, see Sambrook et al., Molecular Cloning A Laboratory Manual, Second Edition, Cold Spring Harbor Laboratory Press, NY, 1989, pp. 15.3-15.113. U.S. Pat. No. 6,150,401, which is incorporated in its entirety herein by reference, also discloses techniques which may readily be adapted to create dominant negative polypeptides to Interleukin 12 p35.

Inhibitors of Interleukin 12 p35 may be antisense compounds, including antisense oligonucleotides, ribozymes and other catalytic oligonucleotides, and inhibitory RNAs including transfected, intracellularly expressed single stranded antisense RNAs or double stranded RNAs, as well as small intefering RNAs (siRNA).

Ribozymes are catalytic RNAs. A number of labs around the world are now using these ribozymes to study gene function in precisely the manner described above most notably in the study of HIV, the AIDS virus, and in cancer research. Ribozymes may be synthetically engineered via the technologies of Ribozyme Pharmaceuticals, Inc. (RPI), Boulder, Colo., to act as “molecular scissors” capable of cleaving target RNA, for example the mRNA encoding Interleukin 12 p35, in a highly specific manner, blocking gene expression. Various types of ribozymes and their uses are taught, for example, in U.S. Pat. Nos. 6,436,644 and 6,194,150.

siRNAs are short double stranded RNAs (dsRNA) which may be designed to inhibit a specific mRNA, for example the mRNA encoding Interleukin 12 p35. PCT publication WO 00/44895 (Kreutzer and Limmer) discloses methods for inhibiting the expression of a predetermined target gene in a cell. Such method comprises introducing an oligoribonucleotide with double stranded structure (dsRNA) or a vector coding for the dsRNA into the cell, where a strand of the dsRNA is at least in part complementary to the target gene. U.S. Pat. No. 6,506,559 discloses and claims gene-specific inhibition of gene expression by double-stranded ribonucleic acid (dsRNA) and is incorporated herein by reference in its entirety. See also PCT publications WO 01/48183, WO 00/49035, WO 00/63364, WO 01/36641, WO 01/36646, WO 99/32619 and WO 00/44914, and Elbashir et al., Functional anatomy of siRNAs for mediating efficient RNAi in Drosophila melanogaster embryo lysate, EMBO J., 2001, 20, 6877-6888. Thus, one of ordinary skill in the art can readily design an inhibitory RNA, such as a dsRNA (e.g., an RNAi or siRNA compound) or a vector coding for the inhibitory RNA, which is capable of inhibiting the nucleotide sequence encoding the Interleukin 12 p35 protein.

Antisense oligonucleotides and antisense oligonucleotide mimetics such as peptide nucleic acid (PNA) and morpholino compounds are preferred antisense compounds. Antisense compounds specifically hybridize with one or more nucleic acids encoding Interleukin 12 p35. Examples of antisense inhibitors of Interleukin 12 p35, as well as various chemical modifications and methods for making and using them are disclosed in U.S. Pat. No. 6,399,379, the contents of which are incorporated herein in their entirety.

The inhibitors used in the present invention may also admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The compounds used in the present invention encompass any pharmaceutically acceptable salts, esters, or salts of such esters, or any other compound which, upon administration to an animal, including a human, is capable of providing (directly or indirectly) the biologically active metabolite or residue thereof. The term “pharmaceutically acceptable salts” refers to physiologically and pharmaceutically acceptable salts, i.e., salts that retain the desired biological activity of the parent compound and do not impart undesired toxicological effects thereto.

The methods of the present invention may also use pharmaceutical compositions and formulations of one or more Interleukin 12 p35 inhibitors. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Pharmaceutical formulations may conveniently be presented in unit dosage form and may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions used in the methods of the invention may be formulated into any of many possible dosage forms such as, but not limited to, tablets, capsules, gel capsules, liquid syrups, soft gels, suppositories, and enemas. The compositions may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions include, but are not limited to, solutions, emulsions, foams and liposome-containing formulations. The pharmaceutical compositions and formulations used may comprise one or more penetration enhancers, carriers, excipients or other active or inactive ingredients.

One of skill in the art will recognize that formulations are routinely designed according to their intended use, i.e. route of administration.

Preferred formulations for topical administration may include those in which the compounds to be administered are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearoylphosphatidyl choline) negative (e.g. dimyristoylphosphatidyl glycerol DMPG) and cationic (e.g. dioleoyltetramethylaminopropyl (DOTAP) and dioleoylphosphatidyl ethanolamine (DOTMA).

For topical or other administration, Interleukin 12 p35 inhibitors used in the invention may be encapsulated within liposomes or may form complexes thereto, in particular to cationic liposomes. Alternatively, inhibitors may be complexed to lipids, in particular to cationic lipids.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. In some embodiments, inhibitors are administered in conjunction with one or more penetration enhancers, surfactants and chelators. Examples of surfactants include fatty acids and/or esters or salts thereof, bile acids and/or salts thereof. Combinations of penetration enhancers may also be used.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the methods of the invention involve use of pharmaceutical compositions containing one or more inhibitors of Interleukin 12 p35 and one or more other agents that function by a non-Interleukin 12 p35 mechanism. Examples of such agents include but are not limited to cancer chemotherapeutic drugs, anti-inflammatory drugs, including but not limited to nonsteroidal anti-inflammatory drugs and corticosteroids, and antiviral drugs. In preferred embodiments, the other agent(s) may be an anti-diabetes drug. In addition to the well known treatment, insulin, which may typically be porcine or human and is typically given by needle injection or pump, there are several types of orally administered treatments for diabetes. Oral hypoglycemics, starch blockers, insulin sensitizers and drugs which decrease the production of glucose by the liver and increase glucose utilization by the tissues are all comprehended by the present invention. Common orally administered drugs for diabetes include insulin, pioglitazone, glimepiride, metformin, rosiglitazone, rosiglitazone/metformin, sulfonylurea, glyburide, glyburide/metformin, glipizide, miglitol, glipizide/metformin, repaglinide, acarbose, troglitazone, and nateglinide. When used in combination, the Interleukin 12 p35 inhibitor and the additional agent may be used individually, sequentially or in combination.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual inhibitors, and can generally be estimated based on EC₅₀s found to be effective in in vitro and in vivo animal models. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the inhibitor is administered in maintenance doses.

Various U.S. Patents and applications have been cited herein. The contents of these documents are incorporated in their entirety herein by reference. A patent application directed to antisense inhibitors of Interleukin 12 p35 was filed on May 7, 2001 (Docket No. RTS-0241) and issued on Jun. 4, 2002 as U.S. Pat. No. 6,399,379; the disclosure of this document is incorporated in its entirety herein by reference.

While the present invention has been described with specificity in accordance with certain of its preferred embodiments, the following examples serve only to illustrate the invention and are not intended to limit the same.

### EXAMPLES

### Example 1

Triglyceride Accumulation Assay:

This assay measures the accumulation of triglyceride by newly differentiated adipocytes. The in vitro triglyceride assay model used here is a good representation of an in vivo model because a time-dependent increase in triglyceride accumulation by the adipocytes has been shown to increase concomitantly with increasing leptin secretion. Furthermore, an increased in triglyceride content is a well established marker for adipocyte differentiation.

Triglyceride accumulation is measured using the Infinity™ Triglyceride reagent kit (Sigma-Aldrich, St. Louis, Mo.). Human white preadipocytes (Zen-Bio Inc., Research Triangle Park, N.C.) are grown in preadipocyte media (ZenBio Inc.) One day before transfection, 96-well plates are seeded with 3000 cells/well. Cells are treated according to standard published procedures with Interleukin 12 p35 inhibitor (in this experiment, 250 nM oligonucleotide) in lipofectin (Gibco). Monia et al., J. Biol. Chem., 1993, 268, 14514-22. Inhibitors are tested in triplicate on each 96-well plate, and the effects of TNF-α, a positive drug control that inhibits adipocyte differentiation, are also measured in triplicate. Negative controls and transfectant-only controls may be measured up to six times per plate. After the cells have reached confluence (approximately three days), they are exposed to differentiation media (Zen-Bio, Inc.; differentiation media contains a PPAR-y agonist, IBMX, dexamethasone and insulin) for three days. Cells are then fed adipocyte media (Zen-Bio, Inc.), which is replaced at 2 to 3 day intervals. On day nine post-transfection, cells are washed and lysed at RT, and the triglyceride assay reagent is added. Triglyceride accumulation is measured based on the amount of glycerol liberated from triglycerides by the enzyme lipoprotein lipase. Liberated glycerol is phosphorylated by glycerol kinase. Next, glycerol-1-phosphate is oxidized to dihydroxyacetone phosphate by glycerol phosphate oxidase. Hydrogen peroxide is generated during this reaction. Horseradish peroxidase (HRP) uses H₂O₂ to oxidize 4-aminoantipyrine and 3,5 dichloro-2-hydroxybenzene sulfonate to produce a red-colored dye. Dye absorbance, which is proportional to the concentration of glycerol, is measured at 515 nm using an UV spectrophotometer. Glycerol concentration is calculated from a standard curve for each assay, and data are normalized to total cellular protein as determined by a Bradford assay (Bio-Rad Laboratories, Hercules, Calif.). Results are expressed as a percent±standard deviation relative to transfectant-only control.

The Interleukin 12 p35 inhibitor employed in this assay is an antisense oligomer, ISIS 138969; SEQ ID NO: 1, and the control (or negative control) employed in this assay is a nonsense oligomer, ISIS 29848, NNNNNNNNNNNNNNNNNNNN SEQ ID NO. 2, where N is a mixture of A, C, G and T. Other antisense inhibitors of Interleukin 12 p35, their synthesis and uses are disclosed in U.S. Pat. No. 6,399,379.

At 250 nM of Interleukin 12 p35 inhibitor, the triglyceride accumulation was reduced by 94% as compared to control. This indicates that differentiation of preadipocytes to adipocytes was inhibited by treatment with Interleukin 12 p35 inhibitor.

### Example 2

Leptin Secretion Assay for Differentiated Adipocytes:

Leptin is a marker for differentiated adipocytes. In this assay, leptin secretion into the media above the newly differentiated adipocytes is measured by protein ELISA. Cell growth, treatment with Interleukin 12 p35 inhibitor and differentiation procedures are carried out as described for the triglyceride accumulation assay (see above). On day nine post-transfection, 96-well plates are coated with a monoclonal antibody to human leptin (R&D Systems, Minneapolis, Minn.) and are left at 4° C. overnight. The plates are blocked with bovine serum albumin (BSA), and a dilution of the media is incubated in the plate at room temperature for 2 hours. After washing to remove unbound components, a second monoclonal antibody to human leptin (conjugated with biotin) is added. The plate is then incubated with strepavidin-conjugated horseradish peroxidase (HRP) and enzyme levels are determined by incubation with 3, 3′, 5,5′-Tetramethylbenzidine, which turns blue when cleaved by HRP. The OD₄₅₀ is read for each well, where the dye absorbance is proportional to the leptin concentration in the cell lysate. Results are expressed as a percent±standard deviation relative to transfectant-only controls.

### Example 3

Hallmark Gene Expression:

During adipocyte differentiation, the gene expression patterns in adipocytes change considerably. This gene expression pattern is controlled by several different transcription factors, including glucose transporter-4 (GLUT4), hormone-sensitive lipase (HSL) and adipocyte lipid binding protein (aP2). These genes play important roles in the uptake of glucose and the metabolism and utilization of fats.

Cell growth, treatment with Interleukin 12 p35 inhibitor and differentiation procedures are carried out as described for the triglyceride accumulation assay. On day nine post-transfection, cells are lysed in a guanidinium-containing buffer and total RNA is harvested. The amount of total RNA in each sample is determined using a RIBOGREEN assay (Molecular Probes, Eugene, Oreg.). Real-time PCR is performed on the total RNA using primer/probe sets for three adipocyte differentiation hallmark genes: glucose transporter-4 (GLUT4), hormone-sensitive lipase (HSL) and adipocyte lipid binding protein (aP2). Expression levels for each gene are normalized to total RNA, and values±standard deviation relative to transfectant-only controls are entered into the database.

The Interleukin 12 p35 inhibitor employed in this assay is an antisense oligomer, ISIS 138969; SEQ ID NO. 1; and the control (or negative control) employed in this assay is an nonsense oligomer, ISIS 29848, NNNNNNNNNNNNNNNNNNNN SEQ ID NO: 2; where N is a mixture of A, C, G and T. Other antisense inhibitors of Interleukin 12 p35, their synthesis and uses are disclosed in U.S. Pat. No. 6,399,379.

At 250 nM of Interleukin 12 p35 inhibitor, aP2 was reduced by 67% and GLUT4 was reduced by 89% as compared to control. This indicates that differentiation of preadipocytes to adipocytes was inhibited by treatment with Interleukin 12 p35 inhibitor.

What is claimed is:

1. A method for inhibiting the differentiation of an adipocyte cell comprising contacting a preadipocyte cell with an effective amount of an inhibitor of Interleukin 12 p35, whereby adipocyte differentiation is inhibited.

2. A method for inhibiting lipid accumulation in a cell comprising contacting a cell with an inhibitor of Interleukin 12 p35, whereby lipid accumulation in the cell is inhibited.

3. The method of claim 2 wherein the cell is a preadipocyte or adipocyte cell.

4. The method of claim 2 wherein lipid accumulation is triglyceride accumulation.

5. A method of treating a disease or condition associated with adipocyte differentiation in a mammal comprising administering to a mammal an effective amount of an inhibitor of Interleukin 12 p35, whereby adipocyte differentiation is inhibited.

6. The method of claim 5 wherein the disease or condition is obesity, cardiovascular disease, metabolic syndrome, diabetes, insulin resistance or cancer.

7. A method of treating a disease or condition associated with excess adipocytes in a mammal comprising administering to a mammal an effective amount of an inhibitor of Interleukin 12 p35, whereby adipocyte differentiation is inhibited.

8. The method of claim 7 wherein the disease or condition is obesity, cardiovascular disease, metabolic syndrome, diabetes, insulin resistance or cancer.

9. A method of treating a disease or condition associated with lipid accumulation in a mammal comprising administering to a mammal an effective amount of an inhibitor of Interleukin 12 p35, whereby lipid accumulation is inhibited.

10. The method of claim 9 wherein the disease or condition is hyperlipidemia, obesity, cardiovascular disease, metabolic syndrome, diabetes, insulin resistance or cancer.

11. The method of claim 9 wherein lipid accumulation is triglyceride accumulation.

12. A method of treating a disease or condition associated with high triglyceride levels in a mammal comprising administering to a mammal an effective amount of an inhibitor of Interleukin 12 p35, whereby triglyceride accumulation is inhibited.

13. The method of claim 12 wherein the disease or condition is hypertriglyceremia, obesity, cardiovascular disease, metabolic syndrome, diabetes, insulin resistance or cancer.

14. Use of an inhibitor of Interleukin 12 p35 in the manufacture of a medicament to inhibit the differentiation of adipocyte cells.

15. Use of an inhibitor of Interleukin 12 p35 in the manufacture of a medicament to inhibit lipid accumulation in a cell.

16. The use of claim 15 wherein lipid accumulation is triglyceride accumulation.


SEQUENCE LISTING (2 sequences)

SEQ ID NO: 1 - DNA, 20 bp, Artificial Sequence
  Antisense Oligonucleotide
ggaacattcctgggtctgga

SEQ ID NO: 2 - DNA, 20 bp, Artificial Sequence
  Antisense Oligonucleotide
nnnnnnnnnnnnnnnnnnnn